(19) United States
(12) Patent Application Publication
(10) Pub. No.: US 2026/0340826 A1
(43) Pub. Date: Oct. 29, 2026

(54) METHOD FOR REFERENCE IMAGE DETERMINATION

(71) Applicant: BRAINLAB SE, Münich (DE)

(72) Inventors: Markus Mayer, Munich (DE); Lukas KOMPATSCHER, Munich (DE); Franz GUM, Munich (DE); Jörg REHS, Munich (DE)

(21) Appl. No.: 19/106,123

(22) PCT Filed: Nov. 23, 2023
(86) PCT No.: PCT/EP2023/082897

§ 371 Date: Feb. 24, 2025

(51) Int. Cl. G06T 7/00; A61B 5/00; A61B 5/08; A61B 5/11; A61B 5/113

(52) U.S. Cl. CPC G06T 7/0014; A61B 5/0816; A61B 5/1127; A61B 5/1128; A61B 5/113; A61B 5/7285; G06T 2207/10016; G06T 2207/30004; G06T 2207/30168

(57) ABSTRACT

Provided is a method for reference image determination. The method includes obtaining a breathing signal derived from patient monitoring data of a patient and selecting a reference image among a plurality of surface images of a patient surface. The plurality of surface images are acquired while acquiring the patient monitoring data. Selecting the reference image includes selecting a surface image as the reference image based on determining that the breathing signal, at the time of image acquisition of the surface image, meets one or more criteria associated with the breathing signal.

### FIELD OF THE INVENTION

The present invention relates to a computer-implemented method for reference image determination, a system, a computer program product, and a computer-readable medium.

### TECHNICAL BACKGROUND

Surface imaging methods are commonly used for patient positioning and monitoring, e.g. for radiation treatment.

As an example, prior to treatment, a combination of surface imaging data and data from medical imaging modalities, such as cone beam CT (CBCT) imaging, may be obtained.

The final treatment position of the patient is usually defined based on the medical imaging data, such as the CBCT data. A suitable reference surface image is selected among the surface imaging data, i.e. an image that is representative of the medical imaging data at the final treatment position.

The reference surface image is later used for patient positioning and monitoring during radiation treatment, e.g. based on surface imaging during the radiation treatment.

For many medical applications, such as the irradiation of breast tumors, the patients are required to perform a deep inspiration breath hold (DIBH). Thus, a suitable reference surface image is one that is representative of medical imaging data, such as a CBCT, acquired during a DIBH.

A challenge is, that medical scans, such as CBCT acquisition, usually take relatively long. In case of CBCT, for example, acquisition may last about 30-60 seconds.

During that time, the patient may move or exhale partly (involuntarily) and it may even require multiple breath holds to acquire a complete scan, such as complete CBCT.

Accordingly, many different surface images can be acquired during (or before and after) a single scan, such as a single CBCT imaging, and not all of them are necessarily suitable reference surface images. It is also not immediately clear which is a suitable reference surface image.

Current methods rely on a person selecting intuitively, among a plurality of potential surface images, a reference surface image to be used as a reference.

This has several disadvantages. Intuitive selection may not be sufficiently reliable and reproducible, particularly where the selection is done in real time, e.g., by triggering surface acquisition at a time that is intuitively considered suitable. This effect can be somewhat mitigated if the reference surface image is selected retroactively, but a large number of surface images then needs to be stored and analyzed after acquisition, which is resource-intensive.

The present invention has the object of providing a method, system, computer program product, and computer-readable medium that allow for overcoming at least some of the above-identified challenges.

The invention can be used for providing information that may be applicable to procedures e.g. in connection with a system for image-guided radiotherapy such as VERO® and ExacTrac®, ExacTrac Dynamic®, each a product of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The invention provides a method, a system, a computer program product, and a computer-readable medium according to the independent claims. Preferred embodiments are laid down in the dependent claims.

The present disclosure provides, among others, a method for reference image determination. The method comprises obtaining a breathing signal derived from patient monitoring data of a patient. The method also comprises selecting a reference image among a plurality of surface images of a patient surface, the plurality of surface images acquired while acquiring the patient monitoring data. A surface image is selected as the reference image in case it is determined that the breathing signal, at the time of image acquisition of the surface image, meets one or more criteria associated with the breathing signal.

### GENERAL DESCRIPTION OF THE INVENTION

In this section, a description of the general features of the present invention is given for example by providing possible embodiments of the invention.

The present disclosure provides a, particularly computer-implemented, method for reference image determination. The method comprises obtaining a breathing signal derived from patient monitoring data of a patient. The method also comprises selecting a reference image among a plurality of surface images of a patient surface, the plurality of surface images acquired while acquiring the patient monitoring data. A surface image is selected as the reference image in case it is determined that the breathing signal, at the time of image acquisition of the surface image, meets one or more criteria associated with the breathing signal.

In other words, meeting the one or more criteria may be a necessary condition for a surface image to be selected as the reference image. It may optionally be a sufficient condition for a surface image to be selected as the reference image, or other conditions may additionally apply.

The method may, in particular, be a computer-implemented method. That is, at least some, in particular all of the steps of the method of the present disclosure may be performed by a computing system, also referred to as data processing system, in particularly, fully automatically, unless otherwise specified.

As will be apparent from the above, the selection of a reference image is carried out based on criteria that are associated with/related to the breathing signal, which in turn is derived from patient monitoring data. As such, an objective selection is carried out. The method allows for selecting a reference image (reference surface) that is objectively more suitable than many other potential reference images in view of selecting the reference image that corresponds to an objectively suitable breathing level of the patient.

This improves reliability and reproducibility, which cannot be ensured by practitioners intuitively selecting image acquisition time or selecting a reference image.

More specifically, as an example, for various medical applications, such as the irradiation of breast tumors, the patients are required to perform a deep inspiration breath hold. For patient positioning, a combination of surface/thermal and CBCT imaging may be used. The final treatment position in this case is defined by the CBCT. It is therefore required to find a surface image that is representative of a CBCT acquired during a DIBH. The challenge is, that CBCT acquisition takes 30-60 seconds while the patient may move or exhale partly (involuntarily) and it may even require multiple breath holds to acquire a complete CBCT, so many different surfaces can be acquired during (or before and after) a single CBCT imaging and it is not immediately clear which is a suitable reference. The reference surface image is later used to monitor the patient position. An additional challenge is that the high number of surface images may require too large amounts of resources, e.g., for storage and/or data analysis.

The method of the present disclosure allows for overcoming at least some of the above challenges.

In the present disclosure, the term “image” is to be understood broadly as the resulting image data of an image acquisition.

A surface image, according to the present disclosure, may be any image that comprises or allows for deriving depth information of a surface, such as the patient surface. For example, a surface image may be an image acquired by a 3D surface camera, a thermal-surface imaging device, a 3D surface scanner, or the like.

The surface represented or depicted by the reference image may be considered to be a reference surface. The reference image is, accordingly, also referred to as reference surface image or reference surface herein.

The breathing signal may be any data that reflects the breathing movement of a patient. As such, it may be a time series or function of time, for example, of values or images. The breathing signal may comprise breathing signal data. Examples for the breathing signal will be given further below.

The breathing signal being derived from patient monitoring data may comprise that the breathing signal, particularly the breathing signal data, is at least partially included in the patient monitoring data, and deriving the breathing signal may comprise selecting the breathing signal (data) among the patient monitoring data. The breathing signal being derived from the patient monitoring data may, alternatively or in addition, comprise that the breathing signal (data) is obtained by a calculation, such as by a model, the calculation taking at least part of the monitoring data as input and calculating and outputting the breathing signal (data).

Examples for the patient monitoring data will be given further below. The patient monitoring data may, optionally, comprise the surface images.

As explained above, the plurality of surface images are acquired while acquiring the patient monitoring data. As the breathing signal is derived from the patient monitoring data, this allows for obtaining surface images associated with a given portion of the breathing signal, e.g., the breathing signal at a given time or in a given time interval.

Thus, for example, for each surface image, the breathing signal, e.g. its value, shape or other characteristics, at the time of image acquisition may be determined. The value, shape, or other characteristics may also, for the sake of brevity, be collectively referred to as the breathing signal associated with the surface image or breathing signal value associated with the surface image. Accordingly, it is also possible, to determine, for a surface image, whether the associated breathing signal (value) meets criteria.

As mentioned above, a surface image can be selected as the reference image in case it is determined that the breathing signal, at the time of image acquisition of the surface image, meets one or more criteria associated with the breathing signal.

In other words, for each of a plurality of surface images, the breathing signal, particularly its value at or shape around the time of image acquisition may be analyzed to determine whether it meets the one or more criteria and, in response to determining that the one or more criteria are met, the surface image may be selected as a reference image.

Criteria may relate to a value of the breathing signal, a shape of the breathing signal, or other characteristics of the breathing signal. Which criteria are used depends, for example, on the nature of the signal. This will be explained in more detail below.

According to the present disclosure, the breathing signal may be a time-dependent value of a parameter representative of breathing level of a patient, the parameter derived from the patient monitoring data. Particularly, it may be a time-dependent value of one single parameter representative of breathing level of a patient, the parameter derived from the patient monitoring data.

As an example, the parameter may be an angle and/or a position of a surface portion or a parameter derived therefrom, the surface portion moving, particularly changing position and/or orientation, when the patient breathes. The breathing signal may then be a time-dependent value representative of an angle and/or position of the surface portion or a value derived therefrom.

A breathing signal may be determined using an axis, for example a vertical axis, and intersections of this axis with the monitored patient surface at a given time (live surface of the patient). This approach brings speed. It reduces or removes any latency between the surface and derived breathing signal. The axis may be defined by a selected point on the patient's surface, e.g. manually selected point. The axis does not necessarily have to be a vertical axis. For example, the main direction of movement of the breathing may be used as the axis.

Reference is also made to WO 2021/004620 A1 providing examples how a breathing signal may be obtained. The method of said disclosure of determining a breathing signal and/or a breathing curve of a patient comprises determining a motion trajectory of a structure associated with at least one body part of the patient, wherein the motion trajectory is indicative, descriptive and/or representative of a respiratory movement of the structure; acquiring surface data representative of a position of a surface region of the patient; computing, calculating and/or determining an intersection of the determined motion trajectory and the acquired surface data; and determining a breathing signal and/or a breathing curve of the patient based on the computed intersection, wherein the breathing signal is indicative of a breathing state of the patient. As one example, the motion axis may describe one or more movement components related to one or more spatial directions the structure is displaced during breathing. Particularly, the motion axis may refer to a main motion axis indicative of a main movement component of the respiratory movement of the structure. For instance, the main motion axis may be determined based on trajectory data using principal component analysis. For instance, the main motion axis may be defined by the first principal component (optionally also the second and/or third principal component) as determined based on analysing the trajectory data using PCA. Further, the motion trajectory of the structure may be determined based on the determined motion axis and/or the determined main motion axis. In this context, the motion trajectory may be defined by and/or may be given by the motion axis and/or the main motion axis.

The patient monitoring data may comprise tracking data. Tracking data may comprise position and/or orientation of a patient surface, particularly of a surface portion that moves, particularly changes position and/or orientation due to breathing, and may be data acquired using common tracking techniques, for example marker-based tracking or marker-less tracking.

Alternatively or in addition, the patient monitoring data may comprise the plurality of surface images and the parameter may be representative of movement of a surface portion of the patient, particularly movement of a landmark of the surface portion and/or movement of a marker placed on the surface portion, due to breathing. Commonly known image processing techniques may be used to derive such a parameter from the images.

Alternatively or in addition, the patient monitoring data may comprise spirometer data. Spirometer data may be used to represent or derive the breathing signal.

Alternatively or in addition, the patient monitoring data may comprise data from pressure and/or stretch sensors. Such sensors may be applied to areas of the patient that deform due to breathing. Thus, the data from pressure and/or stretch sensors may be used to represent or derive the breathing signal.

Alternatively or in addition, the patient monitoring data may comprise data obtained by wearable sensors. For example, wearable sensors may comprise acceleration sensors that allow for determining breathing motion.

In an example, the breathing signal may represent changes of the height of a chest portion of a patient.

Alternatively or in addition to parameter values, the breathing signal may also comprise surface images, either as originally acquired or compressed, particularly a time series of surface images, as will be explained further below.

While this may be good for accuracy, using a breathing signal that is expressed by a time-dependent value of a parameter may allow for a more resource effective method, particularly a method that does not require storing large amounts of data from surface images and may reduce resources used for determining whether the one or more criteria are met.

As can be understood from the above, the breathing signal is representative of the breathing level of a patient. Thus, for example, a change in the breathing level is reflected by a change of the breathing signal. A breathing level may be an amount of inhalation or expiration relative to a reference amount, such as a maximum amount, of inhalation or expiration, respectively. The breathing level may, for example, be reflected in the amplitude of a breathing signal, optionally relative to a reference amplitude, which may be referred to as baseline level. The reference amount or reference amplitude may be selected among values obtained during DIBH.

According to the present disclosure, the plurality of surface images may comprise surface images acquired during one or more breath hold periods, and the reference image may be selected among the surface images acquired during the one or more breath hold periods.

A breath hold period is a period during which a patient holds their breath. For example, as explained above, the patient may be instructed to hold their breath during a CBCT. A patient may also follow a breathing pattern with repeated breath hold periods. A breathing signal may, for example, have an approximation of a plateau during a breath hold period.

As explained above, it is advantageous to select a reference image that has been taken during a breath hold period in case subsequent procedures, where the reference surface is used, also rely on a breath hold period (such as irradiation of the chest), such that a surface image from that time best represents the relevant future state.

According to the present disclosure, selecting the reference image may comprise determining, for at least some of the surface images, a corresponding breathing signal value at the time of image acquisition of the respective surface image, wherein at least one of the criteria described above may be associated with breathing signal values.

As explained above, a surface image is selected as reference image in case one or more criteria are met. Where the breathing signal comprises a set of values at different times, the breathing signal value at the time of image acquisition may be used for selecting a reference image. The method may comprise determining whether the respective breathing signal values meet the criteria. This will be explained in more detail below.

According to the present disclosure, the at least one of the criteria associated with the breathing signal values may be associated with a relation between the respective determined breathing signal value and a reference breathing signal value.

For example, the criteria may be associated with/relate to a difference or a ratio of the breathing signal value and the reference breathing signal value. The reference breathing signal value may be a predetermined breathing signal value or may be a calculated breathing signal value, particularly a breathing signal value that is updated based on prior breathing signal values, e.g., prior breathing signal values of the same breath hold period.

Using a reference breathing signal value may allow for providing even more consistent and reproducible results.

According to the present disclosure, the at least one of the criteria associated with the relation between the respective determined breathing signal value and a reference breathing signal value may comprise at least one of: the determined breathing signal value being equal to the reference breathing signal value, the difference between the determined breathing signal value and the (corresponding) reference breathing signal value having a predetermined value or being within a predetermined interval or exceeding a predetermined threshold value or being below a predetermined threshold value, the ratio of the determined breathing signal value and the (corresponding) reference breathing signal value having a predetermined value or being within a predetermined interval or exceeding a predetermined threshold value or being below a predetermined threshold value.

Using a reference breathing signal value in the above manner may allow for providing even more consistent and reproducible results.

According to the present disclosure, the reference breathing signal value may be derived from a plurality of breathing signal values of the breathing signal derived from the patient monitoring data, particularly obtained from patient monitoring data acquired during (the) one or more breath hold periods.

That is, the reference breathing signal value may not be a fixed or predetermined value. Instead, the reference breathing signal value may be determined on a case-by-case basis. More specifically, it may be derived from breathing signal values derived during one or more breath hold periods, particularly at least the breath hold period during which the plurality of surface images is acquired.

The reference breathing signal value may be a running average of the breathing signal, particularly during one or more breath hold periods.

According to the present disclosure, the reference breathing signal value may comprise a statistical value, the statistical value comprising at least one of:
an average breathing signal value for a predetermined time interval. In particular, the average breathing signal value may be from a time interval during which the surface images are acquired, particularly from a breath hold period during which the surface images are acquired.
a running average breathing signal value, particularly a simple, cumulative, or weighted running average breathing signal value. In particular, the average breathing signal value may be calculated for breathing signal values of the breathing signal during a time when the surface images are acquired, particularly a breath hold period during which the surface images are acquired.
a median of the breathing signal value for a predetermined time interval. In particular, the median may be from a time interval during which the surface images are acquired, particularly from a breath hold period during which the surface images are acquired.
a running median of the breathing signal value. In particular, the median value may be calculated for breathing signal values of the breathing signal during a time when the surface images are acquired, particularly a breath hold period during which the surface images are acquired.
a root mean square breathing signal value.

In an example, the reference breathing signal value, particularly the statistical value, may be calculated individually for each surface image. In other words, different sets of breathing signal values may underly calculation of the reference breathing signal value for different surface images. For example, the breathing signal values obtained up to the acquisition of a respective surface image may be used for calculating the breathing signal. Accordingly, the breathing signal value may, thus, be continuously updated and, accordingly, different for each surface image.

According to the present disclosure, the method may comprise storing the selected reference image, optionally together with at least one of:
a/the corresponding breathing signal value.
a/the corresponding reference breathing signal value.
a value representative of the relation, particularly difference, between a/the corresponding breathing signal value and a/the corresponding reference breathing signal value.
a rating score of the reference image, the rating score reflecting characteristics of a/the determined breathing signal value and/or characteristics of the reference image, the characteristics, for example, comprising a/the relation between breathing signal value and reference breathing signal value and/or image quality of the reference image.
a stability score that is a measure of stability of the breathing signal at the time of acquisition of the surface image and/or in a time interval around the time of acquisition of the surface image, in particular, wherein the stability score comprises a standard deviation of the breathing signal.

Storing the selected reference image allows for future retrieval for different applications where the reference image may be used.

Moreover, the above may allow for improved use of the selected reference image in the intended applications and improved precision. Where a plurality of candidate reference images are stored, this may allow for a final selection that selects a candidate reference image that may not be the top candidate in terms of the one or more criteria used for the initial selection (for example closest to a target breathing signal value) or the most recent candidate reference image, but is better by some other measure, such as has a better rating score or stability score. Finally, it may also help in identifying error sources and/or identifying wrongly selected reference images automatically based on the stored data.

According to the present disclosure, for each newly acquired surface image, it may be determined whether the one or more criteria are met, and, in case the one or more criteria are met, the surface image may be selected as the reference image.

Thus, over time, more than one surface image may be selected as a reference image. Some or all surface images selected as reference images may be stored. This may allow for a future sub-selection as explained above. However, it also requires a large amount of storage.

According to the present disclosure, in case the newly acquired surface image is selected as the reference image and another surface image had previously been selected as the reference image, the newly acquired surface image may replace the previously selected surface image as the reference image.

That is, at any given time, there may only be a single reference image, i.e., the most recent one. This is advantageous because it does not require any subsequent selection among a set of candidate reference images, while yielding good results. Even though potentially the most recent may not be the best reference image, results are still significantly more objective and reproducible than those of known methods. Particularly, the one or more criteria ensure that the reference images always meet some minimum requirement.

A method where a previously selected reference image is replaced by the most recently selected reference image may be referred to as rolling surface reference update.

According to the present disclosure, upon selecting a surface image as a reference image, at least some of previously acquired surface images may be discarded.

Discarding may be carried out in real time. For example, discarding may be carried out concurrently with the surface image acquisition.

Once reference image has been selected, this reference image may be stored. This reference image alone is sufficient for future use. Accordingly, some or all other surface images, optionally including older reference images, may be discarded. This greatly reduces resource usage, such as storage and/or data transmission.

According to the present disclosure, all of the previously acquired surface images (i.e. previous to the selected reference image) may be discarded. That is, the selected surface reference image may be kept, e.g. stored, and all images acquired prior to the selected reference image, particularly all images except for the selected surface reference image, may be discarded. For example, only the most recent selected surface reference image may be kept.

Thus, only one surface image needs to be stored as reference image at any given time, which may be replaced when a more recent surface image is selected as reference image. This reduces resource usage.

As will be described below, there are different ways that reduce resource usage by selectively retaining/keeping and discarding surface images in an objective manner based on technical considerations for the technical application at hand.

For example, according to the present disclosure, the method may comprise determining to discard a previously acquired surface image based on rules, the rules comprising at least one of:
discarding previously acquired surface images when it is determined that a predetermined threshold number of previously acquired surface images is reached. Particularly, it may avoid errors due to slowing down the system with vast amount of data. This may be done without discriminating between reference images and non-reference images or it may take into account whether a surface image had been selected as a reference image, e.g. as explained below.
discarding all previously acquired surface images that have not previously been selected as reference image. Surface images that had not been selected as reference images are likely not going to be used, so it is usually safe to discard these images. It may be possible to retain all reference images or to also discard some of them, as will be explained below.
discarding surface images previously selected as reference image when it is determined that a predetermined threshold number of previously selected surface images is reached. If the number is set at 2 or more, there is at least one, particularly several fallback options should another image that had been selected as reference image not be usable for some reason.
discarding surface images after a predetermined period of time has lapsed. As an example, surface images may be discarded after a given number of breath hold periods or after a given time. The older images are, the more likely it is that they do not properly reflect the current situation, such that data can be cleaner by discarding older images.
discarding the surface images in the order of respective time of image acquisition, starting with the oldest surface image and progressing to more recent surface images. This rule may also apply for images that had previously been selected as reference images.

Other rules are also conceivable, and the rules may be suitably combined. Particularly, scenarios are conceivable where oldest images are discarded first and where a rule is in place ensuring that the discarding of images does not lead to the number of stored reference images dropping below a predetermined number of reference images, which may be one or more than one.

According to the present disclosure, all previously acquired surface images except the most recent reference image or a predetermined number of the most recent reference images may be discarded.

This is an embodiment that works particularly well in terms of resource usage and yields reliable results.

According to the present disclosure, the method may further comprise prospectively defining acquisition points for acquisition of candidate surface images, based on a breathing signal target value. The acquisition points may correspond to different breathing signal values within a tolerance interval around the breathing signal target value. The method may further comprise acquiring, for each of the acquisition points, a corresponding candidate surface image, and selecting, among the corresponding candidate surface images, the reference image. The breathing signal target value, in particular, may correspond to the reference breathing signal value. The selection may, in particular, be carried out automatically.

According to the present disclosure, the candidate surface image associated with the acquisition point that is closest to the target value may be selected as reference surface image.

Alternative ways of selecting a reference image among the candidate surface images may comprise selection based on timing criteria, such as newest reference image, or based on rating or stability scores, or the like, as described further above.

According to the present disclosure, the method may comprise providing an external reference surface, wherein the breathing signal may comprise surface images. The one or more criteria may, in this example, comprise a similarity criterion for a similarity between the surface images of the breathing signal and the external reference surface. The surface images of the breathing signal may comprise at least some of the plurality of the (acquired) surface images or a plurality of derived surface images derived from the plurality of the (acquired) surface images. Derived images may, for example, be compressed and/or converted images. A similarity criterion may be applied to the surface images of the breathing signal, particularly to the similarity compared to the external reference surface. Particularly, the surface image of the breathing signal that has the greatest similarity to the external reference surface, particularly has the smallest root mean square error, RMSE, value, may be determined using the similarity criterion. This image may be selected as reference image.

While verifying the criterion may require more computation than verifying criteria for single value breathing signals, the selection criterion is rather intuitive and works with measured data more directly, rather than carrying out several steps of deriving data. Moreover, precision may be increased.

It may be advantageous to carry out the similarity determination using derived images, such as compressed images, rather than the acquired surface images. This may reduce the required amount computing resources. It is then still possible to select, as reference image, the acquired image that corresponds to the derived image meeting the similarity criterion. Thus, high quality reference images can be selected.

According to the present disclosure, when the breathing signal comprises the (acquired) surface images, the surface image of the breathing signal having the greatest similarity to the external reference surface may be selected as the reference image. Alternatively, when the breathing signal comprises derived surface images derived from (acquired) surface images, the (acquired) surface image from which the derived surface image of the breathing signal having the greatest similarity to the external reference surface was derived may be selected as the reference image.

According to the present disclosure, the external reference surface may be acquired using an imaging device, such as a laser scanner or medical imaging device, that is different from an imaging device used for obtaining the plurality of surface images.

Alternatively or in addition, the external reference surface may be a surface that has a known orientation relative to a LINAC.

According to the present disclosure, the method may comprise acquiring medical image data of a patient over a period of time, e.g., a cone beam CT image or an MR image, during acquisition of the breathing signal and determining the external reference surface based on the outer contour of the patient imaged in the medical image data. Herein, “image data” and “imaging data” are used interchangeably.

When obtaining the external reference surface with the medical imaging device, a good match of the selected reference surface (reference image) with the medical image data can be more easily obtained.

According to the present disclosure, the surface images of the breathing signal may be a subset of the plurality of surface images. Alternatively, the plurality of derived surface images of the breathing signal may comprise compressed images obtained by compressing the surface images from which the derived surface images were derived or a subset thereof.

For example, for preserving computing resources, only a predetermined number or share of the surface images may be included in the breathing signal, or the image signal may not comprise the full resolution surface images, e.g., be compressed. The compression may be such that the resulting compressed images still allow for criterion-matching.

According to the present disclosure, the method may comprise using the reference image and image data obtained from a medical imaging (scan) carried out during acquisition of the plurality of surface images for patient positioning and/or monitoring patient position, for example during radiation treatment, particularly in a chest area, and/or for image registration, or the like. Use (e.g. for patient positioning and/or monitoring patient position and/or for image registration, particularly use during radiation treatment) can be performed during the same treatment session, e.g. on the same day, as the treatment session during which the reference image is determined and/or the medical imaging, e.g. a cone beam imaging, is performed. Alternatively or in addition, the reference image/reference surface can be (re-)used during another session, e.g., on another day, e.g. during a session or on day when no cone beam imaging is performed.

According to the present disclosure, the method may comprise acquiring the patient monitoring data and/or acquiring the plurality of surface images, and/or deriving the breathing signal.

The surface images may be acquired by means of a surface camera, a surface scanner, a thermal-surface imaging device, or the like. Acquiring the surface image data may comprise a continuous acquisition at a predetermined frame rate and/or acquisition at predetermined intervals and/or acquisition triggered automatically by a trigger event.

The monitoring data may comprise at least some of the plurality of surface images. Alternatively, other types of monitoring data may be used, as described above.

Deriving the breathing signal may, for example, comprise processing the monitoring data to obtain breathing signal values, such as height or angle. This may, for example, comprise image processing in case monitoring data comprise image data. As another example, the breathing signal values may already be comprised in the monitoring data, e.g., in case the sensors directly detect the breathing level.

According to the present disclosure, the method may comprise acquiring the plurality of surface images during one or more breath hold periods, during which a patient holds their breath.

According to the present disclosure, the method may comprise carrying out a medical imaging scan during acquisition of the plurality of surface images, for example a CBCT scan.

According to the present disclosure, the breathing signal may be determined continuously for the duration of the medical imaging scan.

According to the present disclosure, selecting the reference image may be carried out concurrently with acquiring the monitoring data, particularly in real time, and/or concurrently with a/the medical imaging scan.

This may be combined, in particular, with discarding older surface images in the manner described further above. In particular, discarding older surface images may also be carried out in real time, e.g. concurrently with the medical imaging scan.

According to the present disclosure, acquisition of the plurality of surface image data may be triggered by a medical imaging system, for example dependent on a start of the medical imaging scan. This allows for obtaining accurate and representative surface images, and accordingly, a more accurate and representative reference image.

According to the present disclosure, the reference breathing signal value may comprise a statistical value, e.g. as described further above, for a/the time interval and the start and end of the time interval may be triggered by a medical imaging system, for example dependent on a start of the medical imaging scan.

Particularly, this may be applied with the methods described above in the context of using the reference breathing signal for selecting the reference image.

The above allows for obtaining an accurate and representative reference breathing signal, and accordingly, a more accurate and representative selection of a reference image based on the reference breathing signal.

The invention also provides a system comprising a computing system configured to carry out and/or control the method of the present disclosure, that is one or more, in particular all of the steps of the method of the present disclosure. In particular, the data computing system may comprise one or more processors configured to perform one or more, in particular all of the steps of the method of the present disclosure.

The system of the present disclosure may further comprise an image acquisition device configured to acquire the plurality of surface images, in particular comprising at least one of a surface camera, a surface scanner, a thermal-surface imaging device. Reference is also made to the above description of the surface image acquisition.

The system of the present disclosure may further comprise a monitoring device, which may optionally correspond to the image acquisition device, configured to acquire the patient monitoring data. Other devices may also be used as monitoring device, as described further above in the context of the monitoring data acquisition.

The system of the present disclosure may further comprise a medical imaging device configured to carry out a medical imaging scan, particularly a CT scan, particularly a cone beam CT, CBCT scan. Reference is made to the above description of the medical imaging.

The invention also provides a computer program product comprising computer-readable instructions which, when the program is executed by a computer, cause the computer to carry out and/or control the method of the present disclosure, that is one or more, in particular all of the steps of the method of the present disclosure.

The invention also provides a computer-readable medium comprising computer-readable instructions which, when executed by a computer, cause the computer to carry out and/or control the method of the present disclosure, that is one or more, in particular all of the steps of the method of the present disclosure.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed at providing suitable reference image data, particularly surface data, for image processing, particularly processing of image data obtained by medical imaging, such as CT scans, to improve position monitoring and positioning. For this reason alone, no surgical or therapeutic activity and in particular no surgical or therapeutic step is necessitated or implied by carrying out the invention.

The features and advantages outlined above in the context of the method similarly apply to the system, the computer program product, and the computer readable medium of the present disclosure.

### DEFINITIONS

In this section, definitions for specific terminology used in this disclosure are offered which also form part of the present disclosure.

### Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n-and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services ™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Registering

The n-dimensional image of a body is registered when the spatial location of each point of an actual object within a space, for example a body part in an operating theatre, is assigned an image data point of an image (CT, MR, etc.) stored in a navigation system.

### Image Registration

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

### Marker

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

### Marker Device

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

### Marker Holder

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

### Reference Star

A “reference star” refers to a device with a number of markers, advantageously three markers, attached to it, wherein the markers are (for example detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star for example features a way of being attached to the object (for example, a clamp and/or a thread) and/or a holding element which ensures a distance between the markers and the object (for example in order to assist the visibility of the markers to a marker detection device) and/or marker holders which are mechanically connected to the holding element and which the markers can be attached to.

### Landmarks

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

### Treatment Beam

The present invention may be utilized in the context of radiation treatment using a treatment beam. The treatment beam treats body parts which are to be treated and which are referred to in the following as “treatment body parts”. These body parts are for example parts of a patient's body, i.e. anatomical body parts.

The present invention relates to the field of medicine and for example to the use of beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

Reference is also made in this respect to the following web pages: http://www.elekta.com/healthcare_us_elekta_vmat.php and http://www.varian.com/us/oncology/treatments/treatment_techniques/rapidarc.

### Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia. The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 schematically illustrates a method according to the present disclosure;

FIG. 2 is a schematic illustration of a system according to the present disclosure;

FIG. 3 schematically illustrates a top and side view of a chest region at different breathing levels, and a breathing curve;

FIG. 4 schematically illustrates a breathing curve;

FIGS. 5a and 5b illustrate exemplary systems in which the method of the present disclosure may be carried out.

### DESCRIPTION OF EMBODIMENTS

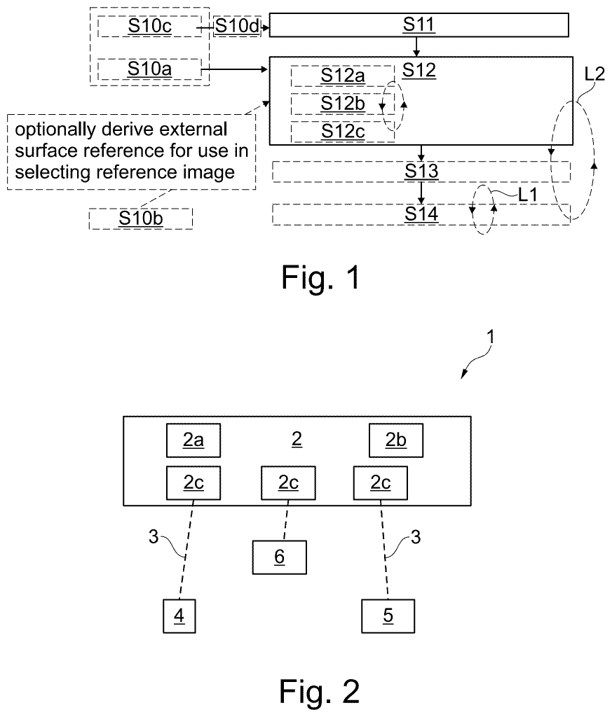

FIG. 1 illustrates exemplary steps of a, particularly computer-implemented, method for reference image determination.

The method comprises, in step S11, obtaining a breathing signal derived from patient monitoring data of a patient.

The method also comprises, in step S12, selecting a reference image among a plurality of surface images of a patient surface, the plurality of surface images acquired while acquiring the patient monitoring data.

A surface image is selected as the reference image in case it is determined that the breathing signal, at the time of image acquisition of the surface image, meets one or more criteria associated with the breathing signal.

As an example, a criterion may be that a breathing signal value associated with a reference image may be within a predetermined range of a reference breathing signal value, such as a moving average of the breathing signal values.

In one example, a surface image may be selected as a reference image in case a single criterion associated with the breathing signal is met, e.g., the criterion described in the preceding passage.

Step S12 of selecting a reference image may comprise, in steps S12a, for an acquired surface image, determining whether the one or more criteria are met and, in optional step S12b, whether additional conditions are met. In case the one or more criteria and optionally additional conditions are met, the surface image may be selected as the reference image in step S12c. These steps may be carried out repeatedly for each newly acquired surface image, indicated by the loop in FIG. 1.

As an example, step S12 of selecting the reference image may comprise determining, for at least some of the surface images, a corresponding breathing signal value at the time of image acquisition of the respective surface image, wherein at least one of the criteria described above may be associated with breathing signal values.

Meeting the one or more criteria may be a necessary condition for a surface image to be selected as the reference image. It may optionally be a sufficient condition for a surface image to be selected as the reference image, or other conditions, e.g., conditions not related to the breathing signal, may additionally apply. If this is the case, the method may comprise, as part of step S12, the optional step S12b of determining whether the other conditions are met. For example, such conditions may relate to quality of images or the like.

The method of the present disclosure may comprise, in optional step S13, storing the selected reference image.

Optionally, the selected reference image may be stored together with at least one of: a/the corresponding breathing signal value; a/the corresponding reference breathing signal value; a value representative of the relation, particularly difference, between a/the corresponding breathing signal value and a/the corresponding reference breathing signal value; a rating score of the reference image, the rating score reflecting characteristics of a/the determined breathing signal value and/or characteristics of the reference image, the characteristics, for example, comprising a/the relation between breathing signal value and reference breathing signal value and/or image quality of the reference image; a stability score that is a measure of stability of the breathing signal at the time of acquisition of the surface image and/or in a time interval around the time of acquisition of the surface image, in particular, wherein the stability score comprises a standard deviation of the breathing signal.

Optionally, in step S14, upon selecting a surface image as a reference image, at least some of previously acquired surface images may be discarded. This may entail that all previously acquired surfaces images, i.e., all images acquired prior to the selected reference image, may be discarded. It may particularly entail that all surface images except for the selected reference image are discarded, including surface images acquired after the selected reference image, optionally unless they are selected as (new) selected reference image.

In one aspect, only the most recently selected reference image is kept, and all other surface images are discarded. In another aspect, a predetermined number, e.g. a number larger than 1, of most recent reference images are kept and all other previously acquired surface images are discarded. For example, any surface images that are not selected as reference images may be discarded, particularly in (near) real time, e.g., while the scan is ongoing.

As part of step S14, a newly selected reference image may replace previously selected reference images, said previously selected reference images being discarded, particularly in real time.

Step S14 can be carried out repeatedly, particularly in a loop L1 together with S12 or in a separate loop L2.

The method may comprise the optional step S10a of acquiring the plurality of surface images, particularly during one or more breath hold periods, during which a patient holds their breath. The step S10a may comprise continuous acquisition of surface images as frames of a video-type image acquisition, repeated acquisition of surface images at fixed intervals, and/or triggered acquisition of candidate surface images.

For example, image acquisition may be triggered at acquisition points, which may, for example, correspond to different breathing signal values within a tolerance interval around a predefined signal target value. For example, for each of the acquisition points, a corresponding candidate surface image may be acquired, and, among the corresponding candidate surface images, the reference image may be selected. The target value, in particular, may correspond to the reference breathing signal value. The candidate surface image associated with the acquisition point that is closest to the target value may then be selected as reference surface image. Other criteria may, alternatively or in addition, be applied for selection.

The method of the present disclosure may comprise optional step S10b of carrying out a medical imaging scan during acquisition of the plurality of surface images, such as a CBCT scan.

The method of the present disclosure may comprise optional step S10c, acquiring the patient monitoring data. Steps S10a and S10b may at least partially coincide, e.g., if the patient monitoring data comprise surfaces images.

The method of the present disclosure may comprise optional step S10d, deriving the breathing signal. The breathing signal may be determined continuously for the duration of the medical imaging scan, for example.

Optionally, step S11 of selecting the reference image may be carried out concurrently with acquiring the monitoring data, particularly in real time, and/or concurrently with a/the medical imaging scan.

Optionally, step S13 of discarding may also be done in (near) real time, i.e., discarding of images may not be done while the imaging is still ongoing, e.g. not only after the imaging has finished.

In FIG. 2, a schematic illustration of a system 1 according to the present disclosure is shown, the system comprising a computing system 2, also referred to as data processing system. The data processing system may comprise at least processing means 2a and storage means 2b, which may comprise temporary memory, e.g., RAM, and/or permanent memory, e.g., ROM. Moreover, optionally the processing system may comprise one or more communication interfaces 2c for receiving and transmitting data via one or more data connections 3. For example, the data processing system may comprise one or more computers.

The computing system 2 is configured to carry out and/or control the method of the present disclosure, particularly as described in the context of the description of FIG. 1.

The system of the present disclosure may further comprise an image acquisition device 4 configured to acquire the plurality of surface images, for example a surface camera or any other imaging devices allowing to acquire surfaces images.

The system of the present disclosure may further comprise a monitoring device 5, which may optionally correspond to the image acquisition device 4, but is shown as a separate device in FIG. 2 as an example. The monitoring device is configured to acquire the patient monitoring data.

The system of the present disclosure may further comprise a medical imaging device 6 configured to carry out the medical imaging scan. The medical imaging device may, for example, be a CT, such as a CBCT imaging device.

In the following, further advantages and examples will be provided.

The present disclosure may entail storing a reference surface (i.e., a reference image) from a surface camera during the acquisition of a cone beam CT, or the like, for example based on a most recent average position, which is reflected in the breathing signal.

Particularly, a rolling reference surface update may be provided, e.g. by replacing an older selected reference surface with a more recent reference surface.

The present disclosure is particularly useful where a reference surface for breath hold procedures is needed, such as during irradiation of a chest region. Such a reference surface is needed for reliably tracking the surface at breathing level during the procedure. Accuracy of the procedure will often rely on an accurate reference surface. This is particularly challenging where medical imaging scans, e.g. a CBCT, rather than for example a single X-ray image, is acquired. It is important that a CBCT (internal anatomy) matches well with the surface, particularly for procedures on structures whose motion is not directly correlated to the breathing level, e.g. tumors in the lung.

Such scans are acquired over a period of time. The present disclosure objectively selects a reference image from surface images acquired during that period of time. This provides an improvement over the art, for example where a person intuitively selects a time during the CBCT and triggers image acquisition of a reference image. Keeping surfaces images from that entire period and a user selecting a reference image afterwards may work better, but it is still not very precise or reproducible and requires a large amount of resources, like storage.

Some embodiments, therefore, allow for reducing resource usage by selecting a reference image during the acquisition and discarding other images, and optionally by replacing older reference images with newer reference images. Thus, overall, a smaller number of surface images needs to be stored.

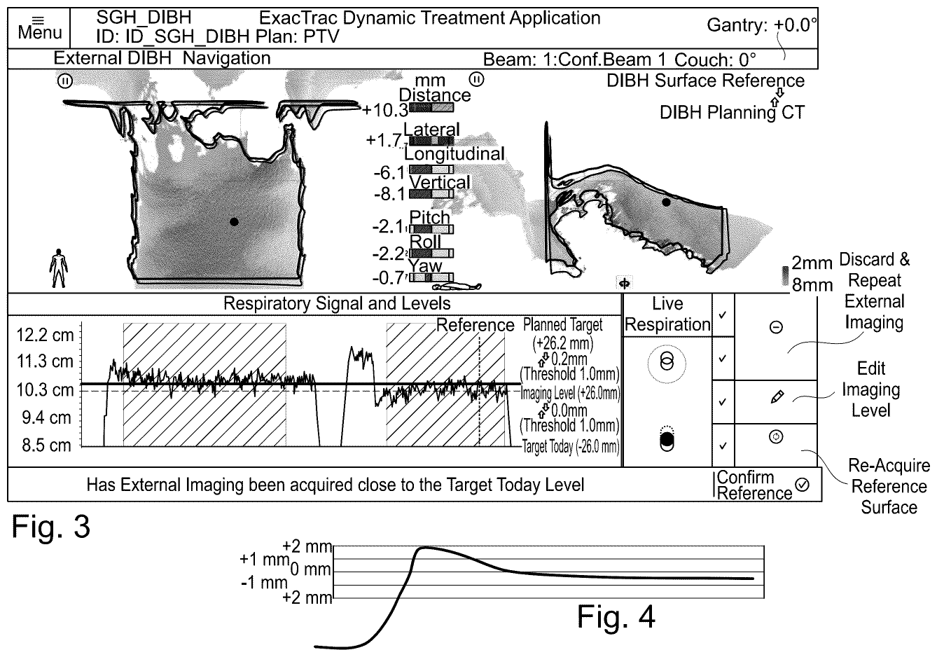

A method according to the present disclosure is illustrated below, also making reference to FIG. 3, where a top and side view of a chest region are shown at different breathing levels.

Moreover, a breathing curve, i.e. a graphic representation of a breathing signal, is shown in FIG. 3, where the signal is a distance calculated to represent breathing level shown as a function of time.

The breathing signal here is obtained over a time that includes DIBH periods, which are reflected roughly as plateaus in the breathing curve.

A vertical line indicates a time when a reference surface image was acquired.

An exemplary method is outlined below. The method employs an average breathing signal value during one or more DIBH periods and the goal is to find a surface reference image where the breathing signal is close to this average breathing signal. example for checking whether the criterion “close to the breathing signal” is met is outlined in the following as well.

Exemplary workflow may be as follows:
BV_now is the current live breathing value (value of the breathing signal), derived from the current live surface Surface_now
The current intermediate surface reference (Surface_ref), i.e. reference image, is stored with the corresponding breathing value (BV_ref) and the current time (T_ref)
   Surface_ref=Surface_now
   BV_ref=BV_now
All breathing values during a medical scan, e.g. CBCT, may be stored (imaging duration may either be selected manually by marking start and stop of imaging, or marked automatically by interface to the medical imaging) as a list (BV1 at T1, BV2 at T2, . . . ).
Every time a new value is added, a running average (BV_avg) of the breathing signal is recalculated.
The following criterion is then applied to the breathing signal:
   IF abs(BV_now−BV_avg)<abs(BV_ref−BV_avg)
   THEN Surface_ref=Surface_now & BV_ref=BV_now
   ELSE n/a
When the medical scan, e.g. CBCT imaging, is finished, the latest intermediate Surface_ref is selected as a surface reference image and may be used for radiation treatment monitoring and positioning, for example.

Fig. 4 shows an example of a breathing curve with candidate acquisition points, here example, surface candidate acquisition points are prospectively defined based on breathing signal target value and a tolerance value (acquisition points for example at mm, at 1 mm, at 2 mm, at 1 mm, at 2 mm). The acquisition points are distributed over tolerable area (here: +/−2 mm). For every acquisition point one surface image may be kept and one of these surface images may be selected as the (final) reference surface. For example the closest one to the breathing signal target value may be selected as the reference surface. Optionally, the selection may be made at run time (real time), e.g. by replacing a respective older reference image with the most recent one.

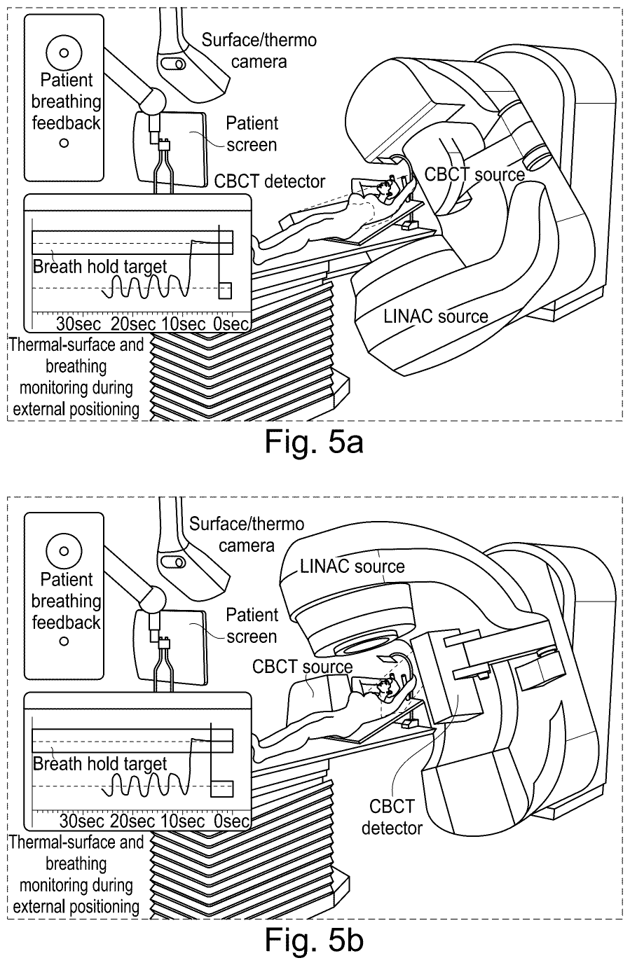

FIGS. 5a and 5b show an exemplary system, in which the method according to the present disclosure may be carried out, in two positions. The system can also be used, subsequently to selecting the reference image, for radiation treatment of a patient.

FIGS. 5a and 5b show a surface/thermo camera 4, which acquires surface images, a CBCT 6 for acquiring medical images, and a radiation source, e.g. LINAC source.

Here, the CBCT and LINAC source are movably mounted and are shown in two different positions in FIGS. 5a and 5b.

Moreover, an optional patient screen and patient breathing feedback are shown, as well as a breathing curve obtained from monitoring data. The breathing curve shows part of a breath hold period (the plateau towards the right of the image).

A thermal surface is also indicated in FIGS. 5a and 5b.

As an example, FIGS. 5a and 5b illustrate the surface and the breathing as they can be observed during external positioning.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1. A method for reference image determination, the method comprising:
obtaining breathing signal derived from patient monitoring data of a patient; and
selecting a reference image among a plurality of surface images of a patient surface, the plurality of surface images acquired while acquiring the patient monitoring data,
wherein the selecting the reference image comprises:
selecting a surface image as the reference image based on determining that the breathing signal, at the time of image acquisition of the surface image, meets one or more criteria associated with the breathing signal.

2. The method of claim 1, wherein the obtained breathing signal is a time-dependent value of a parameter representative of a breathing level of a patient, the parameter derived from the patient monitoring data, and/or wherein the patient monitoring data comprises one or more of: tracking data, the plurality of surface images and the parameter is representative of movement of a surface portion of the patient due to breathing, spirometer data, data obtained from pressure and/or stretch sensors, and/or data obtained by wearable sensors.

3. The method of claim 1, wherein the plurality of surface images comprises surface images acquired during one or more breath hold periods, and wherein the selecting the reference image comprises selecting the reference image among the surface images acquired during the one or more breath hold periods.

4. The method of claim 1, wherein the selecting the reference image comprises determining, for at least some of the plurality of surface images, a corresponding breathing signal value at the time of image acquisition of the respective surface image, wherein at least one criteria is associated with breathing signal values.

5. The method of claim 4, wherein the at least one criteria associated with the breathing signal values is associated with a relation between a respective determined breathing signal value and a reference breathing signal value, wherein the reference breathing signal value comprises a statistical value and the statistical value comprises at least one of:
an average breathing signal value for a predetermined time interval,
a running average breathing signal value,
a median of the breathing signal value for a predetermined time interval,
a running median of the breathing signal value, and/or
a root mean square breathing signal value.

6. The method of claim 5, wherein the at least one criteria associated with the relation between the respective determined breathing signal value and the reference breathing signal value comprises one or more of:
the determined breathing signal value being equal to the reference breathing signal value,
a difference between the determined breathing signal value and the reference breathing signal value having a predetermined value or being within a predetermined interval or exceeding a predetermined threshold value or being below a predetermined threshold value, and/or
a ratio of the determined breathing signal value and the reference breathing signal value having a predetermined value or being within a predetermined interval or exceeding a predetermined threshold value or being below a predetermined threshold value.

7. The method of claim 5, comprising:
deriving the reference breathing signal value from a plurality of breathing signal values of the breathing signal derived from the patient monitoring data.

8. (canceled)

9. The method of claim 1, further comprising:
storing the selected reference image, optionally together with at least one of:
a corresponding breathing signal value,
a corresponding reference breathing signal value,
a value representative of a relation between the corresponding breathing signal value and the corresponding reference breathing signal value,
a rating score of the reference image, the rating score reflecting characteristics of a determined breathing signal value and/or characteristics of the reference image, the characteristics comprising a relation between the breathing signal value and the reference breathing signal value and/or an image quality of the reference image, and/or
a stability score that is a measure of stability of the breathing signal at the time of image acquisition of the surface image and/or in a time interval around the time of image acquisition of the surface image, wherein the stability score comprises a standard deviation of the breathing signal.

10. The method of claim 1, comprising:
for each newly acquired surface image, selecting the newly acquired surface image as the reference image based on determining that the one or more criteria are met.

11. The method of claim 10, wherein the newly acquired surface image is selected as the reference image and another surface image had previously been selected as the reference image, wherein the method comprises:
replacing the previously selected surface image with the newly acquired surface image as the reference image.

12. The method of claim 11, comprising:
upon selecting the newly acquired surface image as the reference image, discarding at least some previously acquired surface images.

13. The method of claim 12, comprising:
determining to discard previously acquired surface images based on rules, the rules comprising at least one of:
discarding previously acquired surface images when it is determined that a predetermined threshold number of previously acquired surface images is reached,
discarding all previously acquired surface images that have not previously been selected as the reference image,
discarding surface images previously selected as the reference image when it is determined that a predetermined threshold number of previously selected surface images is reached,
discarding surface images after a predetermined period of time has lapsed,
discarding the surface images in order of respective time of image acquisition, starting with the oldest surface image and progressing to more recent surface images, and/or
discarding all previously acquired surface images except the most recent reference image or a predetermined number of the most recent reference images.

14. (canceled)

15. The method of claim 1, further comprising:
prospectively defining acquisition points for acquisition of candidate surface images based on a breathing signal target value, wherein the acquisition points correspond to different breathing signal values within a tolerance interval around the breathing signal target value;
acquiring, for each of the acquisition points, a corresponding candidate surface image; and
selecting, among the corresponding candidate surface images, the reference image, wherein the breathing signal target value corresponds to a reference breathing signal value.

16. The method of claim 15, further comprising:
selecting, as the reference image, the candidate surface image associated with the acquisition point that is closest to the target value.

17. The method of claim 1, further comprising:
providing an external reference surface, wherein the breathing signal comprises surface images, wherein the surface images of the breathing signal comprise at least some of the plurality of surface images or a plurality of derived surface images derived from the plurality of surface images, and wherein the one or more criteria comprise a similarity criterion for a similarity between the surface images of the breathing signal and the external reference surface; and
determining, by using the similarity criterion, the surface image of the breathing signal having the greatest similarity to the external reference surface,
wherein the surface images of the breathing signal are a subset of the plurality of surface images, or wherein the plurality of derived surface images of the breathing signal comprise compressed images obtained by compressing the surface images from which the derived surface images were derived.

18. The method of claim 17, further comprising:
selecting, as the reference image, the surface image of the breathing signal having the greatest similarity to the external reference surface image; or
selecting, as the reference image, the surface image from which the derived surface image of the breathing signal having the greatest similarity to the external reference surface was derived.

19. The method of claim 17, comprising:
acquiring the external reference surface using an imaging device that is different from an imaging device used for obtaining the plurality of surface images, and
optionally, the external reference surface is a surface that has a known orientation relative to a LINAC.

20. The method of claim 17, further comprising:
acquiring medical image data of a patient over a period of time during acquisition of the breathing signal;
determining the external reference surface based on an outer contour of the patient imaged in the medical image data;
using the reference image and medical image data obtained from a medical imaging scan carried out during acquisition of the plurality of surface images for patient positioning and monitoring patient position and/or for image registration; and
using the reference image and the image data in a session during which the reference image and the image data are obtained and/or in a different session.

21-22. (canceled)

23. The method of claim 1, further comprising:
acquiring the patient monitoring data; and/or
acquiring the plurality of surface images, comprising:
acquiring the plurality of surface images during one or more breath hold periods, during which a patient holds their breath; and/or
continuously acquiring surface images as frames of a video-type image acquisition; and/or
repeatedly acquiring surface images at fixed intervals; and/or
acquiring, via triggered acquisition, candidate surface images; and/or deriving the breathing signal.

24. (canceled)

25. The method of claim 23, comprising:
carrying out a medical imaging scan during the acquiring of the plurality of surface images; and
continuously determining the breathing signal for a duration of the medical imaging scan.

26. (canceled)

27. The method of claim 1, comprising:
selecting the reference image concurrently with acquiring the monitoring data; and/or
acquiring a plurality of surface image data which is triggered by a medical imaging system dependent on a start of a medical imaging scan,
wherein a reference breathing signal value comprises a statistical value for a time interval and a start and end of the time interval is triggered by a medical imaging system that is dependent on the start of the medical imaging scan.

28. A system for reference image determination, comprising:
a data processing system, the data processing system comprising:
at least one computer comprising a non-transitory memory device and at least one processor configured to communicate with the non-transitory memory device, the non-transitory memory device storing a computer program comprising executable instructions that, when executed on the at least one processor of the at least one computer or loaded onto the at least one processor of the at least one computer, causes the at least one computer to perform a method for reference image determination by:
obtain a breathing signal derived from patient monitoring data of a patient; and
select a reference image among a plurality of surface images of a patient surface, the plurality of surface images acquired while acquiring the patient monitoring data,
wherein the at least one computer is operable to select the reference image by:
selecting a surface image as the reference image based on determining that the breathing signal, at the time of image acquisition of the surface image, meets one or more criteria associated with the breathing signal.

29. The system of claim 28, further comprising:
an image acquisition device configured to acquire the plurality of surface images; and/or
a monitoring device configured to acquire the patient monitoring data,
optionally, the monitoring device corresponds to the image acquisition device.

30. (canceled)

31. The system of claim 28, further comprising:
a medical imaging device configured to carry out a medical imaging scan,
wherein the non-transient memory device is further configured to store the selected reference image.

32. (canceled)

33. A non-transitory computer-readable storage medium storing a computer program comprising computer-readable instructions that, when executed on at least one processor of a computer or loaded onto the at least one processor of the computer, cause the computer to carry perform a method for reference image determination by:
obtaining a breathing signal derived from patient monitoring data of a patient; and
selecting a reference image among a plurality of surface images of a patient surface, the plurality of surface images acquired while acquiring the patient monitoring data.
wherein the selecting the reference image comprises:
selecting a surface image as the reference image based on determining that the breathing signal, at the time of image acquisition of the surface image, meets one or more criteria associated with the breathing signal.
